(19) United States
(12) Patent Application Publication
(10) Pub. No.: US 2006/0062836 A1
(43) Pub. Date: Mar. 23, 2006

(54) Methods of device-assisted drug delivery

(76) Inventors: Stephen G. Carter, Andover, MA (US); Zhen Zhu, Tewksbury, MA (US); Kanu Patel, Derry, NH (US)

Correspondence Address: NIELDS & LEMACK, 176 EAST MAIN STREET, SUITE 7, WESTBORO, MA, 01581, US

(21) Appl. No.: 10/945,593

(22) Filed: Sep. 21, 2004

(51) Int. Cl. A61K 9/70

(52) U.S. Cl. 424/449

(57) ABSTRACT

This invention describes the simultaneous or sequential administration of therapeutic or diagnostic agents using different devices in combination with a chemical formulation that incorporates or uses vasomodulatory chemical agents as part of the drug delivery vehicle. Methods include the addition of various vasodilatory and vasoconstrictive agents to enhance the systemic or localized tissue delivery of therapeutic or diagnostic agents delivered into a body through the use of an apparatus or device.

### FIELD OF THE INVENTION

This invention relates to the transdermal delivery of therapeutic or diagnostic substances using apparatus or devices to assist in the delivery, including iontophoresis, sonophoresis, syringes and needles and micro-needle devices. In particular, this invention describes the enhanced delivery profiles for drug substances when these agents are formulated to include a vasomodulatory chemical agent with the intent to induce either a vasodilatory or vasoconstrictive response in the area of tissue that has been exposed to the drug application (e.g., injection site).

### BACKGROUND OF THE INVENTION

Administration of drug substances through the skin for systemic circulation of the drug or for a localized delivery has been practiced for years through the use of syringes and needles. However, the physical act of introducing a needle into the skin has certain obvious negative reactions including pain and discomfort as well as potential negative side effects to the localized tissue as a result for the trauma of physical disruption due to the relatively large needle penetrating the skin.

Other devices have been developed that also promote the efficiency of transdermal drug delivery, including sonophoresis and iontophoresis. These methods have certain advantages over the syringe and needle method by not breaking the skin, however there are also disadvantages inherent to these technologies, including some skin irritation associated with the adhesives and the tissue disruption due to the energies involved with the delivery. In addition, there are limitations to these technologies related to the speed of drug administration and the associated need to remain attached to an external apparatus during the administration.

The stratum corneum layer of the skin has been identified as the rate-limiting barrier to successful transdermal drug delivery. The technologies listed above addressed this barrier through the energy-assisted movement of drug molecules across this barrier. Others have included the use of different vasoactive chemicals to assist in the optimized delivery of drug molecules, either for localized or systemic delivery (U.S. Pat. No. 5,302,172). The inclusion of vasodilator substances in transdermal delivery vehicle has been described as being useful for enhanced efficiencies in transdermal drug delivery (U.S. Pat. Nos. 5,460,821; 5,645,854; 5,853,751; and 6,635,274).

There are however limits to the abilities of some of these systems, either with or without the use of vasoactive chemicals to achieve successful transdermal drug delivery as a result of the significant barrier presented by the stratum corneum. These drug molecules include those molecules that are larger in physical size and those with significant ionic charges and those with complex quaternary structure.

### SUMMARY OF THE INVENTION

The invention identifies methods to be employed for improving the efficiency of transdermal drug delivery using vasoactive chemicals in the delivery vehicle or in concert with the delivery vehicle. In particular, improvements in delivery efficiency are focused on the inclusion and use of vasoactive chemicals with the devices designed to assist in the passage across the stratum corneum. These devices may include but are not limited to the syringes and needles and microneedle devices with size gauges 30 and larger with smaller needle diameters. This may include any device which uses a physical device used to penetrate the stratum corneum and/or other layers of the skin, then the simultaneous or subsequent or pre-treatment of the injected area with a pharmaceutical formulation containing the active drug molecule and also a vasoactive chemical substance. The vasoactive chemical may be introduced into the injected area, either before, or simultaneous to or following the introduction of the active drug molecule.

The methods described in this invention include compositions of drugs and vasoactive chemical substances in forms that are typical associated with pharmaceutically acceptable formulations sufficient to achieve the desired level of optimized vasodilation or vasoconstriction and also sufficient to achieve the desired pharmacologic delivery of the active drug molecule.

This invention describes the methods necessary for the inclusion of vasoactive chemicals as part of a transdermal drug delivery formulation in concert with any physical skin or stratum corneum penetration device, including but not limited to needles and micro-needles and their associated devices.

The efficiency and the breadth of application of use and result for the prior art has been increased by the present invention to include a broader range of drugs and agents that can be delivered transdermally. These classes of drugs and agents include macromolecular compounds and agents, or other drug molecules whose chemical characteristics were previously precluded from being incorporated into the prior art formulation or configuration for the purpose of transdermal drug delivery. More specifically, the present invention combines functional elements of the transdermal drug delivery system that can perform in more than one functional capacity to achieve the results of delivering a drug or therapeutic or diagnostic agent through the skin and into the bodily fluids. Establishing multi-functioning molecules as part of the delivery system introduces a great degree of flexibility in the system. The molecular size of the delivery complex can be reduced and the chemical characteristics of the delivery complex can be altered. The corresponding reduction in size of the delivery complex permits the consideration of introducing an active drug molecule or agent with a larger molecular weight. This expansion in molecular weight of the active drug molecule may extend to macromolecules (e.g., proteins and peptide fragments). The advantages of the present invention over the prior art have implications for the delivery of active drugs and agents such as large organic molecules including peptides and proteins (e.g., insulin, erythropoietin, interferon, growth hormones). In addition, there are advantages to the incorporation of a vasodilatory substance into the dermal or subcutaneous layer of skin in combination with the active drug molecule, with an improved bioavailability index and also with respect to the speed with which the drug may be introduced into the bloodstream. The addition of a chemical vasodilator could significantly enhance both the efficiency and also enhance the kinetics of the drug uptake.

### DETAILED DESCRIPTION OF THE INVENTION

The invention describes the incorporation of a vasoactive chemical substance in the therapeutic or diagnostic drug formulation that is being delivered transdermally with the assistance of a device, such as, but not limited to, a needle and syringe or a microneedle-type device. The application of drugs and drug substances to the skin, with the desired target of either the localized tissue in and adjacent to the skin or the blood stream for systemic circulation is the goal of this application and invention.

Subcutaneous injections with a standard needle are effective in terms of achieving bioavailability of virtually all drug molecules, regardless of physical size or shape. However, there are disadvantages to this method, including pain, discomfort, infections, and inadvertent bleeding. Despite the limitations of this standard and accepted process, there are advantages such as the avoidance of the stratum corneum layer of the skin, which serves as a primary barrier to the transdermal entry of any substance into the body. As a result, there is a level of consistency with this method that is desired and accepted, the primary issue is the pain and inconvenience of using a syringe and needle.

Microneedles have been developed for the delivery of drug substances, serving to cross the stratum corneum layer of skin, without penetrating deep into the subcutaneous layer. This method also serves advantages with the reduction in discomfort or pain with the injections and also offers the advantage of safety with little concern over cross use or secondary use of the device for purposes other than the original intent. A limitation of this microneedle device and variants of the device is that the low efficiency of delivery of some drugs into the body following deposition of the drug substance into the epidermal and upper dermal layer, for either systemic or localized tissue delivery.

This invention uniquely incorporates the advances made in the microneedle technology and have coupled it with the advances made in device-free transdermal drug delivery technology, for use in the technology of subcutaneous or dermal drug delivery, to elevated the efficiency of the microneedle-assisted process to the level for effective clinical use.

In particular, the use of microneedles offers several focused advantages in the therapeutic or diagnostic fields when the objective is to deliver large molecular weight substances, such as, but not limited to proteins, peptides, DNA, or RNA. These molecules are not good candidates for transdermal delivery because of their inability to cross the stratum corneum as they are large and typically water soluble molecules. Both characteristics make them in opposition to the chemistry and the physical compatibility with the stratum corneum of the skin.

There have been several examples of these molecule classes being delivered through injection means into the skin and tissue surrounding the skin as a method to introduce them into the body. In many instances, this has been acceptable however, the protocol requires a number of injections, as in the case of vaccinations or even for some gene therapy indications. However, in many instances for the treatment of medical conditions and diseases, the need for introducing drug molecules, including proteins and peptides, into the body is required several times each day, which in turn requires the patient or the physician to inject the drug into the subcutaneous layer of the skin, with all of the associated pain and discomfort, such as in the example of insulin-dependent diabetes.

The incorporation of vasoactive substances into the drug formulation has been demonstrated to improve delivery efficiency as either systemic or localized tissue distribution. Injection of microneedle-assisted drugs has avoided many of the negative aspects of standard needle injections but in many cases lacks the efficiency of delivery. This invention describes the method to be used for the incorporation of vasoactive chemical substances into a drug formulation to be delivered into the epidermis, below the stratum corneum, with the purpose and intent to enhance the deliver of the drug substances deposited in that tissue.

The introduction of vasoactive chemicals into a pharmaceutical formulation delivered into the skin tissue, either with the drug substance in the same formulation or separately in advance or subsequent to the injection of the drug substance enhances the delivery of the drug into the blood stream and also deeper into the skin tissue.

The formulation containing the vasoactive chemicals will also contain passive penetration enhancing chemicals, which may be chosen from the class of lipids and lipid-like or lipid-derived molecules, including liposomes and lipid based emulsion and lipid associated hydro-gels. In addition, there may be other chemical agents designed to disrupt or disorganize the architecture of the skin tissue and to promote the penetration of drug substances through the skin.

There are different formats to the use this invention for the delivery of different drugs, depending on the pharmacology profiles desired for that drug and also depending on the interactions of the drug with the other component parts of the delivery enhancement formulation. In one instance, where the drug is stable in the presence of the vasodilator chemical and also in the presence of the other component parts of the formulation, then this may be prepared as a single formulation. The combined drug, vasodilator, penetration enhancing agents, and other formulating chemicals may be prepared in the reservoir of a microneedle device in advance of the application and then administered by applying the microneedle to the skin and injecting the drug.

In contrast, there may be other drug molecules, whose chemistry indicates that it may not be stable for a practical period of time for standard drug formulations, either at room temperature or at a lowered storage temperature when prepared in the presence of the vasodilator(s) or the other component chemicals of the delivery formulation. In this example, then the drug molecule is prepared in a standard solution, which has been demonstrated to maintain the integrity of the drug molecule, and this is inserted into the reservoir of a microneedle device alone. The drug delivery enhancing formulation, containing the vasodilator, penetration enhancer chemicals and other supporting chemicals for the formulation, is prepared as described and introduced into a separate reservoir for a microneedle device. The administration of the drug molecule takes place by first introducing the drug molecule into the skin with the device, followed by the application of the drug delivery enhancing formulation using the separate microneedle device to the same area of skin. The sequence of which formulation to deliver first is determined empirically through experimentation. Alternatively, a novel device composed of microneedles and two separate reservoirs for the two formulations could be applied simultaneously with a single application, through the same microneedles, using partition construction of the device separating the formulations from each other and also from the microneedle portion of the device until the time of application.

In a similar but different application of this technology, the formulations containing vasodilators and penetration enhancing chemicals could be incorporated into devices using iontophoresis and sonophoresis. In these examples, the pharmacokinetic and pharmacodynamic profiles of the drug determine the concentrations used for the enhancing chemicals, such as vasodilators and also penetration enhancers, to ensure that the effect of either the electrical current or the sound waves was enhanced by the presence of the vasodilator enhancing formulation.

The active drug molecule may be included in the same formulation constructed for the delivery of the vasoactive chemical substance, however depending upon several factors, including the possible chemical or micro-environmental lability and stability of the drug substance, the drug substance may be prepared in physiological saline or other formulated chemical vehicle that would be compatible with the subsequent injection into the body using either a needle and syringe and/or with a microneedle device or other device constructed to physically penetrate the stratum corneum and/or other layers of the skin tissue with the purpose of depositing drug substance into the live skin tissue.

Vasoactive drug substances to be included in the chemical formulation may include, but are not limited to: amrinone, L-arginine, bamethan sulphate, bencyclane fumarate, benfurodil hemisuccinate, benzyl nicotinate, buflomedil hydrochloride, buphenine hydrochloride, butalamine hydrochloride, cetiedil citrate, ciclonicate, cinepazide maleate, cyclandelate, di-isopropylammonium dichloroacetate, ethyl nicotinate, hepronicate, hexyl nicotinate, ifenprodil tartrate, inositol nicotinate, isoxsuprine hydrochloride, kallidinogenase, methyl nicotinate, naftidrofuryl oxalate, nicametate citrate, niceritrol, nicoboxil, nicofuranose, nicotinyl alcohol, nicotinyl alcohol tartrate, nitric oxide, nonivamide, oxpentifylline, papaverine, papaveroline, pentifylline, peroxynitrite, pinacidil, pipratecol, propentofyltine, raubasine, suloctidil, teasuprine, thymoxamine hydrochloride, tocopherol nicotinate, tolazoline, xanthinol nicotinate, diazoxide, hydralazine, minoxidil, and sodium nitroprusside. Centrally acting agents include clonidine, quanaberz, and methyl dopa. Alpha-adrenoceptor blocking agents include indoramin, phenoxybenzamine, phentolamine, and prazosin. Adrenergic neuron blocking agents include bedmidine, debrisoquine, and guanethidine. ACE inhibitors include benazepril, captopril, cilazapril, enalapril, fosinopril, lisinopril, perindopril, quinapril, and ramipril. Ganglion-blocking agents include pentolinium and trimetaphan. Calcium channel blockers include amlodipine, diltiazem, felodipine, isradipine, nicardipine, nifedipine, nimodipine, and verapamil. Prostaglandins including: prostacyclin, thrombuxane A2, leukotrienes, PGA, PGA1, PGA2, PGE1, PGE2, PGD, PGG, and PGH. Angiotensin II analogs include saralasin. Other vasodilators include nitroglycerin, labetalol, thrazide, isosorbide dinitrate, pentaerythritol tetranitrate, digitalis, hydralazine, diazoxide, and sodium nitroprusside. This element may serve exclusively as the vasodilation agent or it may also, in addition, serve another function to the delivery complex such as penetration, as the active drug agent, or binding of the delivery complex. One or more vasodilators or chemically modified vasodilators can be used in the delivery complex at any one time for one formulation for the purpose of transdermally delivering an active drug molecule or agent. Typically the concentration of vasodilator to be introduced in the formulation will range between about 0.0005% and about 5%, with the more specific concentration being determined empirically with the desired vasodilator.

Penetration enhancers that may be used as part of the drug delivery vehicle and/or as part of the vasoactive component of the delivery process may include by example only but are not limited to: individual fatty acids or phospholipids or plant extract oils or a plant extract oil/alcohol mix. Suitable fatty acids include by example but are not limited to: linoleic acids, linolenic acids, oleic acids, stearic acids, and myristic acids. Phospholipids include by example but are not limited to: phosphatidylcholine, phosphatidylethanolamine, and phosphatidylserine. Plant extract oils include peanut oil, hemp, barrag, olive oil, sunflower oil, soybean oil, monoi oil and macadamia oil, with olive oil being preferred. Suitable alcohols for the plant extract oil/alcohol mix include ethyl alcohol, isopropyl alcohol, methyl alcohol and witch hazel. Olive oil mixed with isopropyl alcohol is a preferred vegetable oil/alcohol mix. Eucalyptol is a further suitable example of a vegetable oil/alcohol mix. Suitable ratios of vegetable oil: alcohols range from about 5:1 to about 1:10, preferably 1:2. Suitable amounts of plant extract oil or plant extract oil/alcohol mix in the delivery complex range from about 1% to about 66% by weight, more preferably from about 10% to about 33.3% by weight. This component may serve exclusively as the penetrating agent or it may also, in addition, serve another function to the delivery complex such as vasodilation, as the active drug agent, or binding of the delivery complex. One or more penetrating agents or chemically modified penetrating agents may be used in varying quantities or ratios with respect to the other component parts in the drug delivery complex at any one time. The penetrating agent molecule may also serve in any of the other critical functions for the delivery system, including that of active drug molecule, vasodilator, and/or binding agent.

The third element of the delivery complex is the active ingredient. The term “active ingredient” is used herein to indicate any material or composition desired to be delivered transdermally, especially therapeutic drugs and diagnostic agents and especially drug substances that are physically large and difficult to transdermally deliver without the aid of a device. Examples of active ingredients that can be used in accordance with the present invention include but are not limited to: insulin, growth hormone, erythropoietin, interferons, peptide fragments, RNA, DNA and DNA fragments, albumin, keratin, collagens, plasmids, therapeutic proteins, antibodies, Fab fragments of antibodies, Fc portions of antibodies, tolazoline, L-arginine, tocopherol nicotinate, methyl nicotinate, hexyl nicotinate, papaverine, sodium nitroprusside, acetylcholine, lidocaine, tetracine, benzocaine, thiabendazole, hydrocortisone, steroids, hormones, and antisense molecules.

The transdermal delivery formulation may optionally include a fourth primary component in the form of a polymer or a chemical stabilizer molecule. This substance is designed to be compatible with the composition of the remainder of the chemicals in the formulation and will also simultaneously be bio-labile and degrade once the material is in the skin or it may remain on the skin surface as an occlusive barrier. Examples of suitable polymers include but are not limited to: carbopol, pemulen, hydroxyethylcellulose, u-care polymer, and water-soluble gums (e.g., agar, arabic, carob, CMC, carrageenans, ghatti, guar, karaya, kadaya, locust bean, tragacanth, and xanthan gums). The binding agent should be used in an amount ranging from about 1% to about 20% by weight, most preferably 1-2%. The polymer may serve exclusively as the binding agent or it may also, in addition, serve another function to the delivery complex such as vasodilation, penetration, or as the active drug agent of the delivery complex.

### EXAMPLE 1

Oleic acid (5%), gamma linolenic acid (5%), cholesterol (1%), menthol (5%), Lipomulse 165 (2%) and cetyl alcohol (2%) are mixed at 40C for 30 minutes and blended to homogeneous. A separate vessel containing Pemulen (1%) and (10%) propylene glycol is mixed to homogeneity and then added to the first vessel to form an emulsion. A third mixture of tolazoline (1%), papaverine (0.5%) is added in (5%) propylene glycol and (56.5%) deionized water. The mixture is then blended to homogeneity for approximate 20 minutes at room temperature. 100 μg of recombinant human growth hormone is dissolved in (1%) physiologic saline. The growth hormone is added to the main delivery vehicle formulation, blended to homogeneity. A 1 g aliquot is inserted into a reservoir in a 1 cc 29 gauge syringe needle device and injected into the subcutaneous tissue for delivery.

### EXAMPLE 2

Oleic acid (5%), gamma linolenic acid (5%), cholesterol (1%), menthol (5%), Lipomulse 165 (2%) and cetyl alcohol (2%) are mixed at 40C for 30 minutes and blended to homogeneous. A separate vessel containing Pemulen (1%) and (10%) propylene glycol is mixed to homogeneity and then added to the first vessel to form an emulsion. A third mixture of tolazoline (1%), papaverine (0.5%) is added in (5%) propylene glycol and (56.5%) deionized water. The mixture is then blended to homogeneity for approximate 20 minutes at room temperature. 100 μg of recombinant human growth hormone is dissolved in (1%) physiologic saline. The growth hormone is added to the main delivery vehicle formulation, blended to homogeneity. A 1 g aliquot is inserted into a reservoir in a microneedle device, designed to deliver a precise amount of material. The device is placed in contact with the skin and the composite mixture of drug and vasoactive drug delivery formulation vehicle are simultaneously administered.

### EXAMPLE 3

Oleic acid (5%), gamma linolenic acid (5%), cholesterol (1%), menthol (5%), Lipomulse 165 (2%) and cetyl alcohol (2%) are mixed at 40C for 30 minutes and blended to homogeneous. A separate vessel containing Pemulen (1%) and (10%) propylene glycol is mixed to homogeneity and then added to the first vessel to form an emulsion. A third mixture of tolazoline (1%), papaverine (0.5%) is added in (5%) propylene glycol and (56.5%) deionized water. The mixture is then blended to homogeneity for approximate 20 minutes at room temperature. A separate preparation of the active drug molecule (e.g., 0.1% human insulin), dissolved in (1%) physiologic saline is added to the drug delivery formulation in a separate and discrete reservoir in the delivery device. This pharmaceutical formulation (0.1-2 g of each drug delivery vehicle) is applied to a reservoir in a microneedle device, designed to deliver a precise amount of material. The device is placed in contact with the skin and both reservoirs are added to the skin tissue through the action of the device simultaneously.

### EXAMPLE 4

Oleic acid (15%), gamma linolenic acid (5%), cholesterol (2%), menthol (10%), lipomulse 165 (2%) and cetyl alcohol (2%) are mixed at elevated temperatures and blended to homogeneity. A separate vessel containing hydroxyethylcellulose (2%) and propylene glycol is added to the first vessel to form an emulsion. A third mixture of tolazoline (0.1%), papaverine (0.2%), and tocopherol nicotinate (0.5%) is added in propylene glycol and water. The mixture is then blended to homogeneity for approximate 20 minutes at room temperature. A separate preparation of the active drug molecule (e.g., 0.1% human recombinant insulin), dissolved in physiologic saline is added to the drug delivery formulation in a separate and discrete reservoir in the delivery device. This pharmaceutical formulation (0.1-2 g of each drug delivery vehicle) is applied to a reservoir in a microneedle device, designed to deliver a precise amount of material. The device is placed in contact with the skin and the contents of reservoir containing the vasoactive substances are added to the skin tissue. After a 10-minute period, the contents of the reservoir containing the insulin are added to the same location of the skin tissue.

### EXAMPLE 5

Oleic acid (15%), gamma linolenic acid (5%), cholesterol (2%), menthol (10%), lipomulse 165 (2%) and cetyl alcohol (2%) are mixed at elevated temperatures and blended to homogeneity. A separate vessel containing hydroxyethylcellulose (2%) and propylene glycol is added to the first vessel to form an emulsion. A third mixture of tolazoline (0.1%), papaverine (0.2%), and tocopherol nicotinate (0.5%) is added in propylene glycol and water. The mixture is then blended to homogeneity for approximate 20 minutes at room temperature, then the preparation of the active drug molecule (e.g., 0.1% human recombinant insulin), dissolved in physiologic saline is added to the drug delivery formulation and again blended to homogeneity. This pharmaceutical formulation (0.1-2 g) is applied to a reservoir in a microneedle device, designed to deliver a precise amount of material. The device is placed in contact with the skin and the composite mixture of drug and the vasoactive drug delivery formulation vehicle are simultaneously administered.

What is claimed is:

1. In a method of delivering of an active ingredient into the skin with a device that penetrates the stratum corneum layer but not the subcutaneous layer, the step of enhancing the delivery of said active ingredient through said subcutaneous layer by applying to the site of delivery a vasodilator.

2. The method of claim 1, wherein said vasodilator is applied prior to delivering said active ingredient.

3. The method of claim 1, wherein said vasodilator is applied simultaneously with the delivery of said active ingredient.

4. The method of claim 1, wherein said vasodilator is applied after the delivery of said active ingredient.

5. The method of claim 1, wherein said active ingredient is delivered with a microneedle.

6. The method of claim 1, wherein said active ingredient is delivered with a syringe and needle.

7. The method of claim 1, further comprising applying to said site of delivery a penetration enhancing agent.

8. The method of claim 7, wherein said penetration enhancing agent is selected from the group consisting of liposomes, lipid based emulsions and lipid associated hydro-gels.

9. The method of claim 1, wherein said vasodilator is added to a formulation comprising said active ingredient.
